(19) United States
(12) Patent Application Publication
(10) Pub. No.: US 2014/0243220 A1
(43) Pub. Date: Aug. 28, 2014

(54) Metaproteomic Method to Identify Secreted Pattern Recognition Molecules and Adhesive Antimicrobial Factors for Detection of Microbial Agents Eliciting Inflammation in the Human Host

(71) Applicant: J. Craig Venter Institute, Rockville, MD (US)

(72) Inventor: Rembert Pieper, Washington, DC (US)

(73) Assignee: J. Craig Venter Institute, Rockville, MA (US)

(21) Appl. No.: 14/189,541

(22) Filed: Feb. 25, 2014

Related U.S. Application Data
(60) Provisional application No. 61/768,778, filed on Feb. 25, 2013.

(51) Int. Cl. G06F 19/18; G01N 33/68

(52) U.S. Cl. CPC G06F 19/18; G01N 33/6848
USPC 506/8

(57) ABSTRACT

Described herein are methods for analyzing complex host-microbial mixtures. The disclosed methods may be used to diagnosis or prognose in a subject an inflammatory disease, which is caused by or contributed to by microbes.

### RELATED APPLICATIONS

This application claims the benefit of priority to U.S. Provisional Patent Application Ser. No. 61/768,778, filed February 25, 2013; the contents of which are hereby incorporated by reference.

### BACKGROUND

The interactions of microbial human pathogens with their cellular and tissue environments during invasion, most often mucosal epithelial cells in the early phases and subsequently cells contributing to the innate and adaptive immune system, drive most infectious disease processes. Relatively recently, evidence has emerged that many mucosal environments in the human body are not sterile and harbor microbial communities (microbiomes) with which mutualistic relationships are established. In addition, opportunistic pathogens can be tolerated by the human immune system because, at a given time, they do not express virulence factors needed for the invasion process. An example is Staphylococcus aureus, which is a commensal bacterium in the nasal mucosa, but becomes a pathogen when environmental conditions change, e.g. during its transfer to the skin and open wounds¹. Finally, there are interactions between microbes and a given host environment (detrimental to the human host) that do not conform to the principles of infectious disease. Such interactions can lead to immune system perturbations with the result of prolonged inflammation. An example is allergic airway hyper-responsiveness to Aspergillus spp.². The microbial origins for such inflammatory states that may also include eventual auto-immune responses are not always known. One reason for the relative lack of knowledge is the fact that only a minority of human hosts are susceptible to such disease processes. An example is irritable bowel disease and its microbial contributions³. For all of these types of human-microbial interactions, it is clear that a multitude of factors for molecular recognition of the microbes and adhesive defense molecules secreted by the human immune system's cells play important roles in pathogenesis.

Microbiome research has advanced the scientific knowledge as regards the phylogenetic composition of the microbes in specific human body locations, their potential functional roles and their interactions in a more or less complex microbial environment under the influence of the human host. This research has described perturbations of the normal microbial composition in a given body location (dysbiosis), which may result in acute infections or chronic human inflammatory diseases. Such advances have been enabled by next generation genome (Nextgen) sequencing technologies in conjunction with powerful bioinformatics analysis capabilities that filter, process and interpret the massive amounts of relatively short DNA sequence data. Analysis of the data involves assembly of the sequences into contiguous RNA molecules and protein-encoding open reading frames and assignment of gene functions based on sequence alignments of orthologous genes that identify gene functions from already annotated reference genome databases. The largest body of literature has been collected on the human intestinal microbiome⁴ and that of animal models mimicking and testing health and disease conditions for humans⁵. The international Human Microbiome Project has facilitated many of the studies to describe and understand the diversity of the healthy human microbiome and, in several pilot studies, disease-associated microbiome perturbation⁶⁻⁸. One of the most important discoveries in this research field was the extent to which the human immune system is shaped by the intestinal microbiome and, vice versa, the immune system influences the composition of intestinal microbial communities⁹. Developmental aspects of the adaptive immune system in the intestinal mucosa, including the important functions of immune tolerance-promoting regulatory T-cells which produce interleukin-10 and pro-inflammatory CD4 T_{H}17 cells which produce interleukin-17, have been elucidated. Imbalances of the activities of these immune cells has been associated, perhaps in a causative relationship, with irritable bowel diseases⁹. To support the hypotheses of physiological connections between a health-associated microbiome and balance immune system functions, experiments have been conducted using germ-free animals. Such germ-free, newborn animals growing under essentially sterile conditions are not exposed to a microbe-rich environment and do not develop a normal immune system; they have defects in the intestinal vasculature, nutritional and endocrine functions and are more susceptible to infections than conventionally colonized animals^{10, 11}. Discoveries as to whether specific microbial genera or species are associated with dysbiosis/inflammatory diseases and specific microbial species or genera can alleviate the symptoms and counteract the deficiency resulting in inflammatory disease remain challenging, especially in highly complex microbial populations such as those of the human gut and the human skin. In a pioneering study, Mazmanian et al. showed that the human symbiont Bacteroides fragilis protected animals from experimental colitis induced by the opportunistic pathogen Helicobacter hepaticus⁵. Further, the investigators determined the molecule, surface polysaccharide A (PSA), expressed on the surface of the bacterium B. fragilis, which was responsible for the protective and beneficial activities preventing H. hepaticus-induced inflammation in the gut. In addition, they determined that such activities were linked to suppression of interleukin-17 production. Once a B. fragilis strain's ability to produce PSA was abrogated, its colonization of the animal gut no longer resulted in beneficial effects and high pro-inflammatory cytokine levels reoccurred. One of the main cytokine products of regulatory T-cells, interleukin-10, is functionally required to mediate the suppression of interleukin-17 production, thus clearly implicating the balance of T-cell-associated pro- and anti-inflammatory activities into inflammation of the colon. The microbial community in the respiratory tract has also been implicated in protection from or enhanced susceptibility to inflammation and infection. Ichinohe et al. showed that neomycin-sensitive bacteria are associated with the induction of productive immune responses in the lung when challenged with influenza virus A¹². Injection of Toll-like receptor ligands of bacterial origin such as lipopolysaccharides (LPS) and peptidoglycans (PG) rescued the immune system deficiency in antibiotic-treated mice. The cytokines induced by the presence of these neomycin-sensitive bacteria that apparently protected from viral infection were IL-1β and IL-18. The inflammasome activation mediated by NOD-like receptor activities appeared to be important for this regulation of immunity in the respiratory tract¹². However, the bacteria responsible for the activity were not identified.

These studies^{5, 12} and several other investigations have demonstrated that so-called probiotic bacteria establish cross-talk with the human immune system and produce immunomodulatory compounds that participate in the appropriate activation of components of the immune system. Generally, the compounds are structurally diverse microbe-associated molecular patterns (MAMPs)¹³, rather than the equally diverse pathogen-associated molecular patterns (PAMPs)¹⁴, both of which are recognized by mammalian pattern recognition receptors (PRRs). The molecular details as to how the PRRs differentiate between MAMPs which induce innate immunity but also balance danger signaling with immune tolerance and PAMPs which generally induce the innate and, eventually, adaptive immune systems remain to be unraveled. The Toll-like receptors (TLRs), highly evolutionarily conserved and generally considered to be the most important activators of the innate immune system, can be divided into different families (TLR2 to TLR11)¹⁴ that recognize different types of microbial structures including LPS, PG, lipoproteins, cell surface glycoproteins and proteins, oligosaccharides, lipoteichoic acids, and CpG oligonucleotides, all of which are present in bacterial cell envelopes. More extensive traits of bacterial, fungal, viral and parasitic TLR recognition motifs have been published in several review articles ^{14, 15}. The same TLR can recognize different motifs. For example, TLR-2 interacts with lipoarabinomannan expressed by Mycobacteria and with outer membrane porin proteins expressed by various Gram-negative commensal bacteria and pathogens^{14, 15}. Upon engagement of a PAMP, TLRs expressed on macrophages, dendritic cells and other antigen-presenting cells initiate two intracellular TLR signaling pathways, one of which is shared with the IL-1 receptor via activation of MyD88 adaptor protein and results in eventual translocation of the NF-κB transcription factor into the nucleus. Phosphorylated NF-κB activates the expression of multiple cytokine genes, including IL-6, IL-12 and TNF-α¹⁴. The second signaling pathway results in the activation of the TRAF6 adaptor followed by translocation in the nucleus of phosphorylated IRF-3. IRF-3 mainly induces the expression of interferon genes. The second type of PRR is comprised of the NOD-like family of receptors and CARD-helicase proteins¹⁴. Following microbial uptake via endocytic/phagocytic pathways, these proteins recognize microbe- and specifically pathogen-derived molecules in the cytoplasmic compartment of the mammalian host cell. They also activate NF-κB-mediated production of pro-inflammatory cytokines and type 1 interferons¹⁴. Pathogen escape from innate immune recognition is enabled by modulation of bacterial or viral cell surface molecules or by interference with downstream signaling pathways. The third type of PRR is comprised of Type I C-type lectins which can be structurally subdivided into the cell surface macrophage mannose receptors (MMR) and the secreted collectins and the dendritic cell surface Type II C-type lectin receptors of which the DC-SIGN molecule, also called CD209, and langerin are the prototypes¹⁶. These C-type lectins generally recognize carbohydrate structures in a calcium-dependent manner (C for calcium), although some C-type lectin domains are also able to recognize lipids and proteins. Most of the characterized C-type lectins are surface receptors with transmembrane domains, and different types of lectins have different carbohydrate recognition structures. The C-type lectin receptors' main functions appear to be the binding to and subsequent internalization of microbes, which is usually followed by their destruction via the phagosomal killing pathway. Phagolysosomal degradation, in turn, produces microbial antigenic fragments that are presented by dendritic cells and macrophages. Antigen presentation by MHC surface proteins stimulates the adaptive immune system¹⁶. The receptor MMR delivers microbial antigens to early endosomes, while the receptor DC-SIGN delivers antigens to late endosomes and lysosomes. Type I C-type lectins include proteins that are secreted from cells and exist in soluble forms. The characterized prototypes of these lectins, as a group termed collectins, are mannose-binding lectin (MBL), surfactant protein-A and surfactant protein-D^{16, 17}. Collectins appear to assemble into oligomers upon secretion; they are also part of the broader group of molecules called Secreted Pattern Recognition Molecules (SPRMs). A well characterized immune defense pathway is the complement cascade activation via the lectin pathway which is initiated by recognition of a pathogen by MBL, MBL multimerization and recruitment of mannose-binding lectin-proteases (MASPs). MASPs activate components of the complement system resulting in the formation of the membrane attack complex that initiates cytokine release and killing of the pathogen¹⁸. MBL also has innate immunity-modulatory function via interaction with TLR-2 and TLR-6 in phagosomes¹⁹. Surfactant proteins are multi-functional; they are important for normal phospholipid homeostasis reducing fluid tension on the alveolar surfaces as components of the pulmonary surfactant complex, but also play a critical role in pathogen recognition and enhancement of phagocytosis by macrophages²⁰⁻²². These proteins can initiate both pro- and anti-inflammatory activities depending on their interaction with other PRRs such as Toll-like receptors²².

As indicated above, TLRs and SPRMs interact and, upon engagement of PAMPs, such interactions modulate the human immune responses towards the recognized microbes. Models for the complex interactions of TLRs with other recognition molecules are emerging. In the case of fungal pathogens, this involves dectin, a Type I C-type lectin receptor, galectins, integrins, tetraspanins and CD14, a glycosylphosphatidylinositol-anchored co-receptor that can also be released in secreted form²³. Evidence has emerged that SPRMs and TLRs interact with endogenous self-molecules. Recognition of these endogenous “self' proteins appears to amplify autoimmunity and infection processes²⁴. Phagocytes such as macrophages are activated by TLR engagement of danger associated molecular patterns, which are also termed DAMPs or alarmins. An example of a DAMP protein is calprotectin, a protein complex consisting of the proteins S100-A8 and S100-A9 that is associated with acute and chronic inflammatory processes. Calprotectin has been reported to be an agonist of TLR-4²⁴. Heat shock proteins carrying antigen protein fragments also interact with PRRs and constitute DAMPs²⁵. The occurrence of the cross-talk of SPRMs, specifically C-type lectins, and Toll-like receptors has emerged as an important aspect of the recognition of PAMPs (and DAMPs), establishing a balance of immune tolerance and immune activation¹⁷. Secreted, soluble C-type lectins clearly play an important role in the modulation of immune activation upon colonization and recognition with bacterial pathogens but are also targets of immune evasion. In the respiratory tract, surfactant protein-A cannot bind to the lipid A of Bordetella pertussis, which has a terminal trisaccharide sequence shielding it from binding by the collectin²⁶. Surfactant protein-A was also demonstrated to adhere to the Mycobacterium tuberculosis cell surface glycoprotein Apa²⁷. MBL is important for the murine immune responses against Staphylococcus aureus in intravenous and intraperitoneal infection models²⁸. Based on these studies, it is clearly of importance to identify not only membrane-associated pattern recognition receptors but also secreted C-type lectins and other SPRMs.

Galectins (β-galactoside-binding lectins), as aforementioned another family of SPRMs, are involved in complex patterns of interactions with TLRs and CLRs to modulate recognition of PAMPs and subsequently innate immune responses to a pathogen, constitutes another family of SPRMs not anchored in the membrane of immune cells but secreted by them. Galectins have a functional role as DAMPs and receptors for PAMPs²⁹. These proteins are able to cross-link specific ligands, e.g. TLR-2 and TLR6 with dectin-1^{23, 29}. This can also result in cross-linking of ligands on different cell entities and foster immune cell interactions with each other of immune cell-pathogen cell interactions²⁹. The list of DAMPs is ever-increasing and also includes high mobility group box protein 1 (HMGB1), heat shock proteins, interleukin-1α, defensins, annexins and S100 family proteins²⁹. DAMPs play not only a role in innate immune system activation but also in restoration and regeneration of tissues destroyed either by direct insults or secondary effects of innate immune reactions. Many S100 family proteins, such as S100-A8, S100-A9 and S100-A12, are secreted via a non-classical pathway from cells and are expressed and released by various types of phagocytic cells at the sites of inflammation²⁴The S100-A8 and S100-A9 complex is able to sequester zinc and this inhibits matrix metalloproteinases which contributes to its antimicrobial activity³⁰. Additional SPRMs are the ficolins, a group of oligomeric lectins with subunits consisting of both collagen-like long thin stretches and fibrinogen-like globular domains with binding specificity for N-acetylglucosamine and pentraxins, calcium-dependent ligand binding proteins with a distinctive flattened β-jellyroll structure. Ficolins and pentraxins also engage in cross-talk resulting in apparently synergistic effects in innate immune defense and maintenance of immune tolerance³¹.

Finally, a group of antibacterial proteins released into the mammalian intestine from the pancreas and perhaps intestinal epithelial cells appear to represent an evolutionarily primitive form of C-type lectins that also recognize surfaces of bacteria. These are the regenerating islet-derived proteins REG-3-γ (a synonym is pancreatitis-associated protein 1B) and REG-3-α/β (a synonym is pancreatitis-associated protein 1). In mice, REG-3-γ and REG-3-β were found to have distinct activities. REG-3-γ was directly bacteriocidal for Gram-positive bacteria³², whereas REG-3β played a protective role against intestinal translocation of the Gram-negative bacterium Salmonella entericidis³³. REG-3-γ lacks the complement recruitment domains present in other microbe-binding C-type lectins. It was shown that both human and murine REG-3-γ bind to bacterial targets via interaction with PG carbohydrates. It was suggested further that the protein plays a role in maintaining symbiotic host-microbial relationships³². There are also peptidoglycan recognition proteins (PGRPs) with antibacterial activities and functions in innate immunity without lectin domains³⁴. These PGRPs inhibit an intracellular step in peptidoglycan biosynthesis, in E. coli and B. subtilis, by binding to a two-component regulatory system (CpxAR and CssRS, respectively) and constitutively activate these, thus exploiting a stress response pathway of the bacteria to kill them³⁴. Different immunity-influencing functions are attributed to a cysteine-rich protein family member, resistin. Resistin is a systemic immune-derived pro-inflammatory cytokine with a low Mᵣ targeting both leukocytes and adipocytes and also a recognition protein interacting with TLR-4 and competing for its binding with bacterial LPS³⁵. TLR-4 serves as a receptor for pro-inflammatory effects of resistin in human cells, not by cooperative binding but competitive binding to a TLR-4 on the surface of phagocytic cells. The trefoil factors represent a protein family with low Mᵣs secreted by mucus-secreting enterocytes (goblet cells) into the intestinal milieu^{36, 37}. Trefoil peptides are ectopically expressed adjacent to areas of inflammation within the gastrointestinal tract and may play an important role in both maintaining the barrier function of mucosal surfaces and facilitating healing after injury³⁶. Goblet cells also secrete mucins known as molecules having a barrier function protecting the intestinal mucosa from invasion by microbial organisms. There is evidence that virulence factors of microbial pathogens such as enterohemorrhagic E. coli recognize mucins and degrade them to facilitate invasion³⁸. Glycoprotein GP340 is an example of such a mucin; it is calcium-binding and attributed a role in the defense against bacterial pathogens in the intestine and in the lungs, apparently via cooperative activities with surfactant protein-D³⁹.

While the binding characteristics of SPRMs and other proteins that recognize pathogen surface structures or interact with co-receptors have been studied extensively, there is substantially less evidence that these recognition molecules interact with commensal bacteria mediating immune tolerance. For example, it is known how the PSA molecule produced by B. fragilis protects the mammalian host from the virulence-causing effects of H. hepaticus. PSA mediates a change of balance in pro-inflammatory IL-19 and anti-inflammatory IL-10. But the innate immunity mechanism that precedes differential T-cell activation and likely implicates binding of PRRs and SPRMs to the PSA polysaccharide structure remains to be elucidated. Methods that enable the simultaneous identification of SPRMs that bind to surfaces of or even invade cells members of a microbial community including pathogens are needed. We describe an innovative multi-step approach to isolate microbial species or enrich a microbial sub-community by fractionating populations of cells and subsequent identification of SPRMs binding to such microbial populations. The emphasis of the method is on SPRMs, although membrane-bound PRRs may also be identified with this method if they exist in proteolytic forms maintaining binding to the microbial surface structure. The SPRMs may include proteins that invade the bacterial cell envelope. An example for this capability is the observation that PGRPs enter the cell envelope at cell division sites and interact in the periplasmic space with regulatory proteins³⁴.

In sum, many diseases and conditions are caused or influenced by a complex interplay between a mammalian host and microbial species that colonize the host. The microbial organisms may influence whether immune defenses respond to non-self- and self-molecules and to what extent such immune defenses are protective and beneficial or harmful and detrimental for the host.

Methods are needed to identify the species or genera part of such host-associated microbial communities that activate the immune system and cause protective and beneficial versus harmful and detrimental effects.

### SUMMARY

Featured herein are innovative methods for analyzing complex host-microbial mixtures. These methods utilize a combination of separation techniques, metaproteomic analysis techniques and data interpretation to define the colonizing microbes' beneficial versus detrimental activities towards its host.

The methods may include one or more of the following steps: (1) fractionating a complex host-microbial mixture to obtain insoluble cellular and sub-cellular aggregates; (2) purifying microbes in the aggregates to near-homogeneity (on the species or genus level) or enriching for a microbe containing fraction in which mammalian host proteins that are not bound to the microbes are nearly absent, but may include mammalian host proteins bound to microbial cell surface or cell envelope structures; (3) lysing microbes in the fraction; (4) performing a shotgun proteomic analysis of the bacterial lysates; (5) performing a meta-proteomic mass spectrometry data analysis; and (6) performing a biological analysis of the molecular and cellular functions of the mammalian proteins identified from the purified/enriched microbial sample to assess whether these proteins are involved with a host pro-/anti-inflammatory, pro-/anti-apoptotic or an infection-associated response.

In certain embodiments, the combination of protein sequence databases for the computational searches is replaced by an in silico assembled protein fragment sequence database directly derived from a metagenomic sequence analysis performed from the same sample source that underwent metaproteomic analysis.

In other embodiments, the meta-proteomic analysis includes computational searches of a bacterial protein sequence database derived from a specific microbial genome and of a protein sequence database representing the mammalian host genome or a combination of such protein sequence databases derived from several reference bacterial genomes in combination with the sequence database representing the mammalian host genome;

These methods can be used to understand fundamental processes of host-pathogen and host-commensal interactions, because the mammalian proteins that bind to or invade the cell envelope of microbial cells have important roles in microbial recognition, immune defense via signaling to various cells of the innate and adaptive immune system, immune tolerance via signaling to various cells of the innate and adaptive immune system, and antimicrobial activities.

These methods may also be used to diagnose and prognose certain inflammatory diseases, because the profile of Secreted Pattern Recognition Methods (SPRMs) and adhesive antimicrobial factors such as cationic antimicrobial proteins and peptides (CAMPs) identified in the context of specific commensal microbes and opportunistic pathogens may be indicators of an inflammatory process suggesting the identified microbe's involvement in the disease process.

These methods may be particularly useful for diagnosing or prognosing inflammatory diseases, for example, chronic inflammatory, autoimmune and infectious diseases for which to date, the microbial contributions to the disease process have not been well understood.

These methods are also useful for diagnosing an infectious disease, which is associated with multiple opportunistic pathogens, where it is not immediately obvious which of the opportunistic pathogens is causing the infectious disease. Examples include urinary tract infections, respiratory tract and pulmonary infections, skin and wound infections, gastro-intestinal tract infections and chronic inflammatory diseases. Chronic inflammatory diseases include those of the liver and gastro-intestinal tract (irritable bowel diseases, gastric ulcers, non-alcoholic fatty liver disease, colon cancer), non-healing wounds (e.g., after burns and in diabetic patients) and lungs (chronic obstructive pulmonary disease, sarcoidosis, cystic fibrosis, asthma, chronic bronchitis).

In summary, the method described herein may identify the causative microbial agent(s) for a disease process where the simplified paradigm of “a single pathogen causes an infectious, inflammation-associated disease process” does not apply.

### BRIEF DESCRIPTION OF THE DRAWING

FIG. 1 is a schematic of certain methods described in the following examples. FIG. 1A illustrates the isolation of a bacterial species from a low bacterial complexity sample source (an example is the gnotobiotic piglet model for which metaproteomic data are described in the examples). The bacteria are isolated via centrifugation of a Percoll density gradient⁴⁰ from a host-derived specimen containing a bacterial community and host cells and cell products. Following isolation of the density gradient fraction enriched in the bacteria (e.g. F1), this fraction is subjected to oil immersion microscopy. The lysate is subjected to SDS-PAGE (Coomassie Brilliant Blue stain) analysis to visualize proteins contained in the lysate. FIG. 1B shows the separation of microbes from a stool specimen containing a complex murine distal gut microbial community. Numerous density gradient centrifugation fractions can be isolated (e.g. F1-F10) which are each enriched in distinct subsets of the microbial community. Fractions may be subjected to oil immersion microscopy (e.g. F2 and F5) and lysed. The lysates of fractions isolated from a density gradient fractionation of a murine stool specimen are analyzed by SDS-PAGE to visualize the proteins derived from the lysates). FIG. 1C shows both lysates from the procedure in (A) and (B) being subjected to metaproteomic analysis including protein denaturation and digestion, peptide enrichment, liquid chromatography tandem mass spectrometry (LC-MS/MS) and MS computational analyses using a number of specifically selected protein sequence databases. In this process, the identified peptides are assigned to proteins (and species) or origin based on the quality of peptide-spectral matches (PSMs). The PSM process is automated by algorithms that the MS data search software tools consists of, e.g. Mascot⁴¹. Spectral counting is a process that provides semi-quantitative data on the abundances of the proteins present in the specimen.

FIG. 2 is a schematic depicting the metaproteomic analysis focused on the mammalian host protein identifications and the pertinent biological interpretation of the results. This schematic is in part based on the results provided in Table 1 and in part on literature data describing additional secreted (microbial) pattern recognition molecules (SPRMs) and adhesive antimicrobial factors that were not identified in the data provided in Table 1. The metaproteomic analysis includes computational searches with a customized microbial protein sequence database and a publicly available mammalian host database. Many microbial proteins and typically only a few mammalian host proteins are identified from the LC-MS/MS analysis. This is expected because only a few mammalian host proteins bind to cell surface structures and invade the cell envelope of the microbe. The number of intracellular microbial proteins is comparatively large because the cells are lysed during the sample preparation process. Eight different families of SPRMs that bind to microbial cell surface or envelope structures are shown: PGRP (peptidoglycan recognition proteins), galectins, TFF (trefoil factors), C-type lectins (collectins), ficolins, resistins (cysteine-rich domain proteins), pentraxins, mucins (extensively glycosylated and sulfated glycoproteins). Resistins and some C-type lectins, a larger family of proteins, have been described to have antibacterial properties and could also be defined as adhesive antimicrobial factors.

### DETAILED DESCRIPTION

### Definitions

As used herein, the following terms and phrases have the meanings described below.

“Diagnosis” and “diagnostic method” refer to any method that provides information regarding the presence, nature and/or cause of an infection in a subject. For example, diagnostic methods can provide information regarding the presence of a gastrointestinal tract infection, the extent of the infection, the identity of an infectious agent colonizing a subject's gastrointestinal tract and/or the nature of the host response to this colonization.

“Host” refers to a mammal, for example a human.

“Host protein” refers to a protein, which a mammalian subject or host secretes, for example into its gastrointestinal tract.

“Host-microbial mixture” refers to a biological sample, which contains microbes and host proteins.

As used herein, “inflammatory disease refers to an inflammatory, autoimmune or infectious disease, which is caused or contributed to by a microbial pathogen. Examples include infections of the urinary tract, respiratory tract, skin and wound infections, gastro-intestinal tract infections and chronic inflammatory diseases, such as irritable bowel disease, gastric ulcers, non-alchoholic fatty liver disease, colon cancer, non-healing wounds, chronic obstructive pulmonary disease, sarcoidosis, cystic fibrosis, asthma and chronic bronchitis.

“LC-MS” or “LC-MS/MS” refers to a process in which one or more consecutive liquid chromatography (LC) separation steps is performed to decrease peptide complexity in the sample prior to MS analysis.

“MS/MS” refers to the tandem mass spectrometry mode where the information content for peptide identification is derived from the peptide ion mass-to-charge ratio (m/z) (MS¹ analysis mode) and subsequently generated m/z values of fragment ions with amino acid sequence information (MS² analysis mode).

“Metaproteomic” refers to a proteomic analysis of a mixture of species using an appropriate mass spectrometer (MS) to generate MS data and searching the MS data with a compilation of protein sequence databases that represent at least some of the species in the mixture.

“m/z value” refers to the mass-to-charge ratio of a peptide which can be determined experimentally in a mass spectrometric measurement and predicted in silico from a database.

“Microbe” refers to a microorganism, such as a bacteria, fungi, protest, virus or prion.

“Sample” refers to a biological sample obtained from a host or a preparation made from a such a biological sample.

### Methods

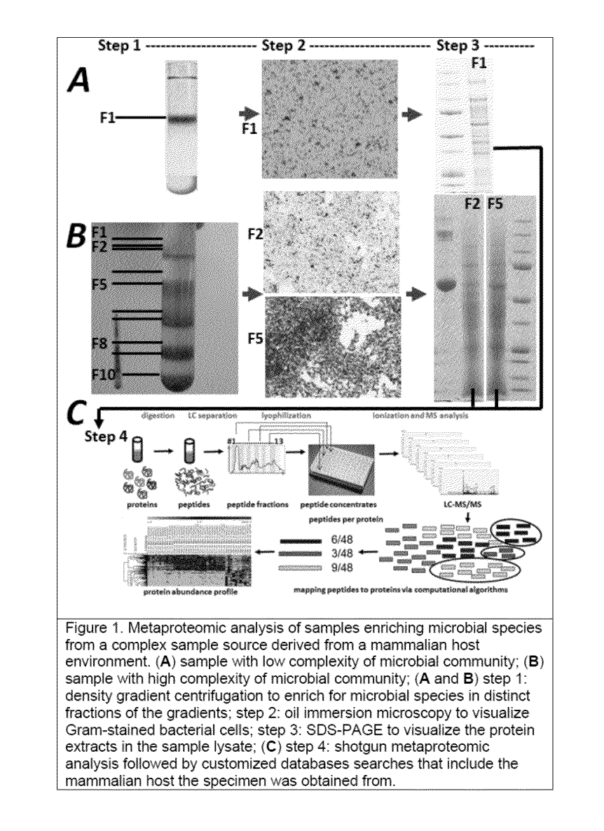

The specimens subjected to metaproteomic analysis may be derived from a mammalian subject and may include, for example, the following sample types: a stool sample, a urine sample, a sputum sample, a saliva sample, a bronchoalveolar lavage fluid sample, a swab from an open skin or wound exudate, a vaginal, nasopharyngeal swab or a tissue biopsy sample from an endoscopy or colonoscopy procedure. Any of these specimens may contain a mixture of microbial species and cells as well as extracellular molecules derived from the mammalian host organism under study. The specimens are typically frozen immediately after recovery from the mammalian host organism. The freezing of the specimens ensures that no extensive protein degradation occurs during their storage prior to the metaproteomic analysis. Here, we provide two example of the sample preparation process for such metaproteomic analyses (FIG. 1). These two examples pertain to stool specimens. However, the application of this method is, as indicated above, not limited to stool specimens. The first sample preparation method pertains to the isolation of a bacterium, enterohemorrhagic Escherichia coli, causing an infectious disease in a gnotobiotic animal in order to model a disease process that does usually not occur in humans (or animals) that are colonized due to exposure to the birth canal of the mother and a microbe-rich environment. Gnotobiotic animals have an intestine essentially sterile because the newborn animals are born via Caesarian Section and kept under aseptic conditions so that they are not colonized with either the mother's microbiota during and after birth or the environment's microbiota after birth. Once these animals are inoculated with a gastro-intestinal pathogen, they may develop a disease that does not develop under normal conditions due to protective microbiota and a rapidly developing host immune system. The second sample preparation method pertains to the isolation of a more complex microbial fraction from a murine stool sample (with a very diverse microbial community), which also contains undigested food products, mucosal cells and cell debris and many molecules secreted into the intestine from the pancreas, bile, liver, blood and intestinal cells). Depending on the microbial sample complexity prior to the separation of the microbes, the samples have different levels of complexity after the microbial fractionation and separation of other materials as illustrated in FIG. 1.

For each type of sample, a density gradient fraction may be re-fractionated to decrease the sample complexity further. For example, a sample initially separated in an iodixanol density gradient may be separated further in a Percoll gradient or size exclusion chromatography. A few or all of these fractions may be processed describing the metaproteomic analysis procedure described herein. Following the isolation of one or more fractions of interest, the metaproteomic analysis begins with a protein extraction step that may be limited to solubilization of cell surface proteins without lysis of microbial cell walls (e.g. the extraction with 1 M NaCl and 0.1% Triton X-100), with partial cell lysis (e.g. the use of methods that apply physical forces such as sonication, bead-beating, exposure to high pressure devices, vacuum-drying/grinding in a mortar) or with more complete cell lysis (combining physical forces with enzymes digesting microbial cell walls such as lysozyme, mutanolysin, lysostaphin, and fungal cell wall chitinases and glucanases). A solution that promotes cell lysis and protein solubilization buffers is typically used. The protein extract can be concentrated by precipitation in an organic solvent (e.g. acetone/5% trichloroacetic acid) or by concentration in a membrane filter device that retains and concentrates proteins but removes small molecules including peptides via filtration). The protein concentrate can be treated by a process, which results in the enzymatic digestion of all proteins; a good example is the FASP method which could be used in combination with a variety of enzymes generating short peptides of ˜5-20 amino acids from the proteins. These enzymes includes trypsin, chymotrypsin, endoproteinase GluC, endoproteinase ArgC and endoproteinase LysC. The digestion process requires between 5 and 100 μg total protein in the extract. Depending on the LC-MS/MS workstation, between 200 and 2,000 proteins can be identified. The protein digests may be frozen at −80° C. prior to LC-MS/MS analysis. A typical LC-MS/MS analysis is described in the following publications^{43, 44} and also in the Examples provided below.

Briefly, the LC-MS/MS analysis first results in separation of peptides in acidified acetonitrile gradients at low flow rates (100-100 nl/min) and direct injection of peptide effluents into an MS source, typically a nano-electrospray source, where the peptides are ionized and enter the mass analyzer. The mass analyzer generates MS and tandem MS spectra that are recorded on a detector. The data acquisition typically occurs in a MS data-dependent mode and the combination of parent ion masses and the corresponding tandem MS fragmentation spectra are used computationally assign peptide spectral matches (PSMs), using a software tool such as Mascot⁴¹ that contains the algorithms necessary to acquire high confidence PSMs. This process entirely depends on databases with protein sequence information that are searched against the entirely of the mass spectral data obtained in a LC-MS/MS run. As indicated in the detailed experimental section, such databases differ and may rely on protein sequence information of a single annotated genome, a combination of annotated genomes or an assembled metagenomic database from a microbial DNA sequencing project that does not attempt to limit open reading frames (encoded proteins) to those that represent complete sequences (from start to stop codon). In all cases, the searched database would include the protein sequences for the mammalian host organism.

Mascot and other software tools score the confidence in peptide identifications, and thus protein identifications for all peptides part of a given protein. Mascot also identifies peptides unique to a given protein sequence among all sequences part of the database. While the summed-up Mascot scores on the protein level provide important information, these unique peptide sequences and their scores are most suitable to identify the microbe(s) present in the analyzed sample.

Mammalian-host derived proteins may also be identified and the Mascot scores for those may be interpreted in the same way as for the microbial proteins. High score levels on the protein level and, in particular, high score levels for the unique peptides that are identified provide confidence in the correct identification of such host proteins. The entirety of the host proteins and their known or predicted functions are evaluated to determine whether the microbial species identified causes effects associated with inflammation, apoptosis, activation of the innate immune system, activation of the adaptive immune system, adhesion to virulence factors of pathogens, antimicrobial effects and tissue regeneration.

All publications, including GI and GenBank Accession numbers mentioned herein are hereby incorporated by reference in their entirety as if each individual publication or patent was specifically and individually indicated to be incorporated by reference. In case of conflict, the present application, including any definitions herein, will control.

The invention, now being generally described, will be more readily understood by reference to the following examples, which are included merely for purposes of illustration of certain aspects and embodiments of the present invention, and are not intended to limit the invention.

### EXAMPLE 1

### Proteomic Analysis of Interactions Between Shiga Toxin-Producing E. coli and the Intestinal Host Environment in Gnotobiotic Piglets

Escherichia coli O157:H7, an enterohemorrhagic E. coli strain of the O-antigen type O157 and the flagellar antigen type H7, constitute the inoculum of gnotobiotic piglets (10⁸ cells). Disease develops in the animals over a period of several days, and diarrheal samples are isolated surgically from the distal gut of a gnotobiotic piglet after the animal was euthanized. The bacterial cells (sometimes more than 1×10⁹ cells) are recovered from the piglets' gut contents, repeatedly washed with PBS and purified via density gradient centrifugation with an isotonic 65% Percoll solution at 14,500×g for 30 min at 4° C.⁴⁵. Percoll solutions result in self-generated optic density gradients during the centrifugation step and allow separations of cells and organelles, e.g. bacterial cells, organelles such as bacterial membrane vesicles and mammalian subcellular organelles, with inherently different optic densities.

FIG. 1A shows a bacterial fraction enriched in a density gradient centrifugation step. Other density gradient procedures could also be applied. Examples are the use of iodixanol gradients described in a previous publication⁴³ and sucrose density gradients.

The bacterial cells are re-suspended in 1 ml of TTE lysis buffer (25 mM Tris-OAc, pH 7.8, 0.05% Triton X-100, 5 mM Na-EDTA, and benzamidine and AEBSF in 1 mM concentrations) to process samples via shotgun proteomics. Samples may be frozen at −80° C. after supplementation with chicken lysozyme (150 μg/ml) and agitation at 20° C. for 1 h. Partially lysed cells are further disintegrated and proteins solubilized by sonication followed by nucleic acid degradation (DNAse I and RNAse at 5 μg/ml) and lysate agitation for 1 h at 20° C. The supernatant and insoluble pellet for each sample are separated by centrifugation at 16,100×g, and the pellet fraction re-extracted with a solution including 2.5 M NaBr. The supernatant may be fractionated further, e.g. by analytical SEC column (G3000-SWXL; 7.8 mm×30 cm; TOSOH Bioscience, USA). Proteins are chromatographically separated in PBS supplemented with 0.01% Triton X-100 into fractions representing the Mᵣ segments >280 kDa, 280-80 kDa and 80-10 kDa. These fractions, containing roughly 60-100 μg protein, are each subjected to digestion with trypsin using a method termed filter-aided sample preparation (FASP)⁴². Briefly, each of the four fractions for a given sample is concentrated in a Microcon YM-10 membrane filter unit (10 kDa Mᵣ cut-off; Millipore, Billerica, Mass.). Twenty μl of a 1 M DTT stock solution and 12 μl of a 10% SDS stock solution are added to denature proteins for 3 minutes at 95° C. Following alkylation, proteolytic digestion (trypsin/bacterial protein ratio of 1:30 to 1:50) is performed at 20° C. overnight. Filtrates containing the peptide mixture are collected by centrifugation at 14,000×g and rinsed three times with 500 mM and once with 50 mM NH₄HCO₃ to recover the protein digestion mixture. Samples were lyophilized. Experimental details for the procedures presented here were published previously⁴³.

### EXAMPLE 2

### Proteomic Analysis of a Human Stool Sample

A mammalian stool sample is weighed (˜1-3 g) and thawed. Cold homogenization buffer (PBST) is added at a 15 ml/g ratio. PBST consists of 100 mM sodium phosphate pH 7.8, 50 mM NaCl and 0.05% Triton X-100. The sample is manually homogenized with a spatula and then stirred overnight at 4° C. The homogenate is filtered through 100 um nylon sieve at 4° C. The insoluble material is discarded (typically enriched in undigested, organic material derived from food products). The filtered material is subjected to centrifugation at 900×g for 15 min at 4° C. The supernatant is retained on ice. The pelleted material is repeatedly extracted using PBST at a 1:7 volume ratio (pellet/PBST buffer) and homogenized by gentle pipetting, similar to a procedure previously published⁴⁶. After approximately re-extractions, all supernatants are combined, and the pellet is discarded. The pellet can be weighed to record the ratio of stool material solubilized with this extraction step versus that remaining insoluble after centrifugal separation at 900×g. The combined supernatants should contain most of the distal gut (stool) bacteria unless they are strongly associated with undigested, insoluble food materials. The microbe-enriched extract is centrifuged at 10,000×g for 15 min at 4° C. in a JA 20.1 rotor to pellet the microbes. The pellet weight is recorded to assess the ratio of enriched microbes compared to the weight of the entire stool sample. The supernatant contains smaller particles including proteins, nucleic acids, polysaccharides, phospholipids and possibly viruses. This supernatant may be retained for further analysis of viral contents. The microbe-enriched extract is resuspended and centrifuged three times to remove any soluble, loosely microbe-associated materials (e.g. polysaccharides from extracellular matrix) and other contaminants. This microbe-enriched extract is used for density gradient centrifugation, as shown in FIG. 1B, and is subjected to further fractionation using density gradient centrifugation protocols. Here we describe the application of iodixanol (Optiprep™) gradient centrifugation.

The iodixanol gradient is prepared in SW60 tubes. The protocol essentially follows one described by the Optiprep™ manufacturer (Axis Shields, Norway)⁴⁷. Stock solutions are prepared first. Gradient stock solution 1 consists (GSS-1) of one volume part of the IODX buffer (3 M NaOAc, 300 mM HEPES, 30 mM MgCl₂, pH 7.8) and five volume parts of Optiprep™ solution. The iodixanol concentration of GSS-1 is 50%. Gradient stock solution 2 (GSS-2) contains 125 mM sucrose, 0.5 M NaOAc, 50 mM HEPES, 5 mM MgCl₂, pH 7.8. GSS-2 can be prepared by six-fold dilution of IODX buffer and addition of the sucrose. To make the gradient layers, GSS-1 and GSS-2 solutions stored at 4° C. are used in different ratios to generate intermediate iodixanol density solutions, one 1 ml pipette tips are used to make these gradient solutions. 792 μL of GSS-1 and 208 μL of the cell lysate sample are combined, transferred into a high speed gradient centrifugation tube (e.g. 11×60 mm) and mixed gently pipetting up and down ˜10 times. This mixture has 40% iodixanol. An ultracentrifuge (set temperature at 18° C.) is switched on and a vacuum is generated to adjust the temperature. Four gradient solutions in 15 mL Falcon tubes from GSS-1 and GSS-2 stocks are prepared: GSS-1 and GSS-2 are combined at a 4:2 ratio; GSS-1 and GSS-2 are combined at a 3:3 ratio; GSS-1 and GSS-2 are combined at a 2:4 ratio; GSS-1 and GSS-2 are combined at a 1:5 ratio. Approximately 650 μL of the 4:2 mixed GSS-1/GSS-2 solution is gently layered on top of the sample dilution. This is followed by layering 650 μL of the 3:3 mixed GSS-1/GSS-2 solution, 650 μL of the 2:4 mixed GSS-1/GSS-2 solution mix and 650 μL of the 1:5 mixed GSS-1/GSS-2 solution. In this order, the iodixanol gradient steps are 33.3%, 25%, 16.7% and 10%, respectively. The gradient tube is balanced with another tube, and 3.6 mL maximal volume of the tube is not surpassed to avoid spillage during centrifugation. The rotor ready (SW60) with all six adaptors is prepared, the vacuum of ultracentrifuge released and the speed set at 50,000 rpm, a centrifugation time of 3 hours, and the vacuum started again. Once the vacuum is at 250, centrifugation begins. Following end of the centrifugation step, the gradient layers in the tubes are checked visually, visible bands are marked, and 1 ml pipette tips are used to aspirate different layers from the top. All gradients layers may contain microbes, depending on the complexity of the sample. Each gradient layer is diluted with an at least 10-fold volume of PBST and spun at maximal speed in a micro-centrifuge tube bench-top centrifuge. The spin is repeated again, if the pelleted microbes do not form a solid pellet. The supernatants are eventually discarded. The pellets may be subjected to an additional round of centrifugation to further enrich microbial species in different gradient layers, for example by changing the steepness of the gradient or by changing the gradient buffer. The pelleted microbial materials may be frozen at −80° C. The microbial pellet is re-suspended in a 1.4 ml TTE-LM) buffer. The term pertains to the buffer constituents (25 mM Tris-HCl, 5 mM EDTA, 0.05% Triton X-100, 50 μg/ml lysozyme and 25 μg/ml mutanolysin). 1 mM AEBSF and 1 mM benzamidine are added to inhibit proteolytic digestion. A microbial sample is homogenized by vortexing several times, incubated at 4° C. overnight, and rigorously vortexed on the next day. The sample is subjected to sonication (in a Misonex 3000 water bath sonicator at the amplitude 8 in 15 30 sec on/30 sec off cycles by intermittent cooling on ice. 10 mM MgCl₂ is added to the lysate followed by addition of 5 μg/ml DNAse, 5 μg/ml RNAse and 5 mM DTT. The suspension is incubated at room temperature for 30 min by gentle shaking to degrade all nucleic acids. The sample is spun at maximal speed in a micro-centrifuge tube bench-top centrifuge for 15 min. The supernatant is retained, and the pellet is resuspended in the FASP digestion buffer^{42, 43} including 0.1% SDS. This sample is vortexed, heated for 3 min at 95° C., and vortexed again. 10 mM MgCl₂ is added to the lysate followed by addition of 5 μg/ml DNAse, 5 μg/ml RNAse and 5 mM DTT. The suspension is incubated at room temperature for 30 min by gentle shaking to degrade all nucleic acids. This pellet-derived sample is spun at maximal speed in a micro-centrifuge tube bench-top centrifuge for 15 min. The first supernatant (derived from the sonication-mediated lysis in TTE-LM buffer) and the second supernatant (derived from the heat-mediated lysis in FASP digestion buffer) are combined for tryptic digestion of the protein mixture by the Filter-Aided Sample Preparation (FASP) protocol.

### EXAMPLE 3

### Subsequent Methods Shared by the Protocols of Example 1 and Example 2: Metaproteomic Analysis

The FASP protocol applies a Microcon filter device (MW cutoff 10,000), trypsin is added at a 1:50 ratio, as described⁴². The protein digestion mixture recovered from the filtrate of FASP processing is lyophilized and reconstituted in 50 μl 0.1% formic acid. Twenty μl of the sample iss subjected to reversed phase C₁₈ LC-MS/MS analysis on an Agilent 1200 solvent delivery system coupled to the nano-electrospray ionization source of an LTQ-XL ion trap mass spectrometer, Thermo Electron LLC). The peptide separation is performed on a BioBasic C₁₈ column (75 μm×10 cm; New Objective, Woburn, Mass.). The LC-MS/MS instrument workflow, the experimental and data analysis parameters has been previously described in Pieper et al., PLoS One 6:e26554 (2011), which is incorporated by reference in its entirety.

The instrument was calibrated prior at the beginning of each day LC-MS/MS experiments were performed with 200 nmol human [Glu¹]-fibrinopeptide B (M.W. 1570.57), verifying that elution times with a CH₃CN gradient varied less than 10% and that peaks representing ion counts had widths at half-height of <0.25 min, signal/noise ratios >200 and peak heights >10⁷. Following quality control and calibration of the LTQ-XL mass spectrometer, loading a 20 μl urinary precipitate lysate sample was followed by trapping and wash (salt removal) of the peptide mixture on a C₁₈ trapping cartridge at a flow rate of 0.01 ml/min for 3 min. Peptides were eluted from the C₁₈ cartridge and separated on the C₁₈ column with 122 min binary gradient runs from 97% solvent A (0.1% formic acid) to 80% solvent B (0.1% formic acid, 90% AcCN) at a flow rate of 350 nl/min. Spectra were acquired in automated MS/MS mode, with the top five parent ions selected for fragmentation in scans of the m/z range 350-2,000 and with a dynamic exclusion setting of 90 sec, deselecting repeatedly observed ions for MS/MS. All peptide fractions from a given urinary precipitate lysate sample were run consecutively on the LC-MS/MS system. The LTQ search parameters (+1 to +3 ions) included mass error tolerances off 1.4 Da for peptide precursor ions and ±0.5 Da for peptide fragment ions. The search engine used for peptide identifications was Mascot v.2.3 (Matrix Science). Search parameters allowed one missed tryptic cleavage, and were set for oxidation of methionine residues as a variable modification. The protein sequence databases to be searched depend on the specific sample type analyzed in the project. It is essential to customize the protein sequence databases.

Protein sequence databases for proteomics analyses (Example 1): The protein sequence database consists of the E. coli 0157:H7 EDL933 protein sequence database (another protein sequence database derived from a different E. coli 0157:H7 strain whose genome was sequenced and annotated could also be used) and of the Sus scrofa protein sequence database in the RefSeq database of NCBI to search for present piglet host proteins if the piglet was the gnotobiotic species subjected to the infection protocol.

Protein sequence databases for proteomics analyses (Example 2): The protein sequence database would consist of a customized “microbiome” database derived from a metagenomic sequencing project and the mammalian host protein sequence database (e.g. the RefSeq human database downloaded from NCBI). To construct the metagenomic database for the analyzed “microbiome”, the JCVI-LIMS provides bar-coded tracking of samples, users, reagents, instruments and material transfers for the Illumina (HiSeq, MiSeq) sequencing pipeline. The processes are customized based on lab protocols, sample tracking, instrumentation and sequencing platform. The sequence data is tracked at the lane level. Data is transferred to a NFS mounted file system in real time with off-instrument signal processing performed on the JCVI grid. After barcode deconvolution, the sample is subjected to GenomeQC analysis, which provides run statistics (raw bases, filtered bases, trimmed bases, total reads, bar-graph of QC20 bases) and screened with BLAST against standard (NT, NR) and custom project dependent databases. With each material transfer, the source and destination containers, reagents, instruments and users involved are recorded via bar-code scanning. Samples and freezer inventories are tracked using wireless PDAs. Extensive support is provided for tracking reagents. Sequencers, fluid handling robots and print-and-apply stations are integrated with the JCVI-LIMS. This tight integration with instruments eliminates the need for users to interact with instruments, thus reducing the opportunity for errors and allowing imposition of the process control. Data processing and QC includes data transfer to the data center for further processing and QC reporting as soon as it is available. Users can monitor quality and browse run data in near real-time allowing the QS group to quickly detect anomalies and optimize processes. For details of the entire analysis process is published process

Protein sequence databases for proteomics analyses (Example 3): This is an example for which experimental methods were not described. If the microbial community investigated is derived from the enriched microbial fraction of a urinary pellet donated by a mammalian subject, the database would consist of microbial species known to colonize the urinary tract. Lactobacillus delbrueckii, Lactobacillus jensenii, Lactobacillus gasseri, Corynebacterium urealyticum, uropathogenic Escherichia coli, Peptoniphilus asaccharolyticus, Klebsiella pneumonia, Klebsiella oxytoca, Streptococcus pneumoniae, Prevotella intermedia, Anaerococcus vaginalis, Staphylococcus epidermidis, Proteus mirabilis, Pseudomonas aeruginosa, Finegoldia magna, Enterococcus faecalis, Enterococcus faecium, Morganella morganii, Enterobacter hormaechei or Ureaplasma urealyticum. In addition, the Homo sapiens protein sequence database in the RefSeq database of NCBI are searched to identify human proteins if the urinary pellet was derived from a human donor.

### Example of a Dataset Resulting in the Conclusion of Invasive, Pro-Inflammatory, and Innate Immunity-Activating Host Responses to an Opportunistic Bacterial Pathogen.

TABLE 1
Proteins identified from a bacterial fraction isolated from the intestinal tract of a
gnotobiotic piglet infected with Shiga-toxin producing E. coli strain 86-24 (STEC).

| Protein name | A/E+ | A/E− | Functional role | Origin | Short name |
|---|---|---|---|---|---|
| Hemoglobin subunit alpha | ++ | − | oxygen transport | blood | HBA1 |
| Hemoglobin subunit beta | ++ | − | oxygen transport | blood | HBB1 |
| Hemoglobin subunit epsilon | ++ | − | oxygen transport | blood | HBE1 |
| Regenerating islet-derived | ++ | ++ | Innate immune defense, | GI epithelium | REG3G, |
| protein 3-gamma | | | antibacterial-Gram (+), | | c-type lectin |
| | | | anti-apoptotic, PRR | | |
| Lithostathine isoform 1 | ++ | ++ | innate immune defense, | GI epithelium | REG1A, |
| | | | causes E. coli | pancreas | c-type lectin |
| | | | aggregation, anti-apoptotic | | |
| Glycoprotein gp340 | ++ | ++ | Innate immune defense, | GI epithelium | DMBT1, |
| | | | target of StcE proteases, | | glycoprotein |
| | | | PRR | | |
| Resistin | + | ++ | Pro-inflammatory, competes | Adipocytes | RETN, |
| | | | with bacterial LPS | GI epithelium | cysteine-rich |
| | | | for TLR-4 binding | | |
| Proteoglycan 3-like protein | − | + | Potentially involved in | Eosinophils | PRG3, |
| LOC 100625180 | | | cytotoxic and | | c-type lectin |
| | | | cytostimulatory activities | | |
| Tyrosine-protein kinase | + | + | Kinase partner for the | Colonic | JAK1, |
| JAK1 | | | interleukin (IL)-2 receptor | epithelium | phosphoprotein |
| Eosinophil peroxidase | + | + | Microbicidal enzyme, | Eosinophils | EPX, heme+ |
| | | | secretion induced by | GI | glycoprotein |
| | | | PRG3 family protein activation | epithelium | |
| Hypothetical protein | + | + | Guanosine 5′-monophosphate | N.D. | — |
| LOC100154068 | | | oxidoreductase domain | | |
| Trefoil factor 3 (intestinal) | + | + | Epithelial cell regeneration, | Goblet cells | TFF3, |
| | | | innate immune defense | GI epithelium | disulfides |
| Hypothetical protein | + | − | Trypsin-like serine | N.D. | — |
| LOC100522363 | | | protese domain | | |

A/E+ and A/E− (+, evidence of expression of proteins associated with the formation of attachment and effacement lesions; −, no evidence of expression of proteins associated with the formation of attachment and effacement lesions). Expression of the A/E lesion-associated virulence factors is associated with damaged to the mucosal walls that can lead to enterohemorrhagy (and intestinal tissue damage as well as more sever systemic symptoms); the data are based on the expression of the intimin (Tir) and the intimin receptor (Eae); ++,+, −: significant peptide-spectral matches >8, <8 or none; Mascot percolator q-value <0.01; PEP value <10⁻⁴; PRR, pattern recognition receptor; N.D. not determined. The LC-MS/MS data were searched with the Sus scrofa (pig) protein sequence database encompassing the entire genome.

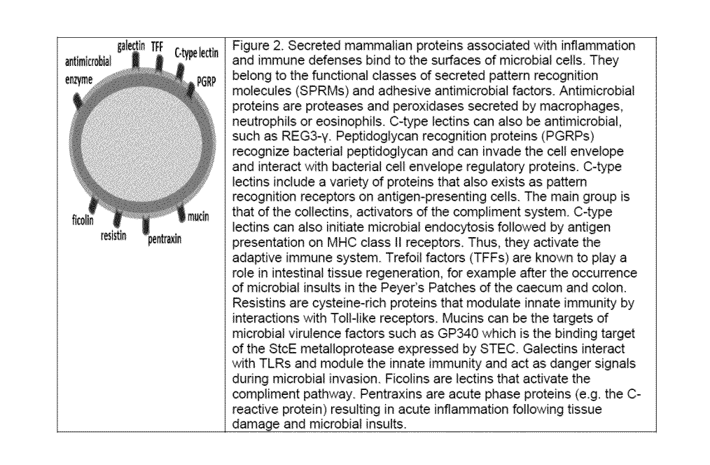

The mammalian proteins identified here represent the majority of protein families presented in FIG. 2.

### REFERENCES

1. Kooistra-Smid M, Nieuwenhuis M, van Belkum A, Verbrugh H. The role of nasal carriage in Staphylococcus aureus burn wound colonization. FEMS Immunol Med Microbiol 2009; 57:1-13.
2. Greenberger P A. Chapter 18: Allergic bronchopulmonary aspergillosis. Allergy Asthma Proc 2012; 33 Suppl 1:S61-3.
3. Bolino C M, Bercik P. Pathogenic factors involved in the development of irritable bowel syndrome: focus on a microbial role. Infect Dis Clin North Am 2010; 24:961-75, ix.
4. Gill S R, Pop M, Deboy R T, Eckburg P B, Turnbaugh P J, Samuel B S, Gordon J I, Relman D A, Fraser-Liggett C M, Nelson K E. Metagenomic analysis of the human distal gut microbiome. Science 2006; 312:1355-9.
5. Mazmanian S K, Round J L, Kasper D L. A microbial symbiosis factor prevents intestinal inflammatory disease. Nature 2008; 453:620-5.
6. Nelson K E, Weinstock G M, Highlander S K, Worley K C, Creasy H H, Wortman J R, Rusch D B, Mitreva M, Sodergren E, Chinwalla A T, Feldgarden M, Gevers D, Haas B J, Madupu R, Ward D V, Birren B W, Gibbs R A, Methe B, Petrosino J F, Strausberg R L, Sutton G G, White O R, Wilson R K, Durkin S, Giglio M G, Gujja S, Howarth C, Kodira C D, Kyrpides N, Mehta T, Muzny D M, Pearson M, Pepin K, Pati A, Qin X, Yandava C, Zeng Q, Zhang L, Berlin A M, Chen L, Hepburn T A, Johnson J, McCorrison J, Miller J, Minx P, Nusbaum C, Russ C, Sykes S M, Tomlinson C M, Young S, Warren W C, Badger J, Crabtree J, Markowitz VM, Orvis J, Cree A, Ferriera S, Fulton L L, Fulton R S, Gillis M, Hemphill L D, Joshi V, Kovar C, Torralba M, Wetterstrand K A, Abouellleil A, Wollam A M, Buhay C J, Ding Y, Dugan S, FitzGerald M G, Holder M, Hostetler J, Clifton S W, Allen-Vercoe E, Earl A M, Farmer C N, Liolios K, Surette M G, Xu Q, Pohl C, Wilczek-Boney K, Zhu D. A catalog of reference genomes from the human microbiome. Science 2010; 328:994-9.
7. Peterson J, Garges S, Giovanni M, McInnes P, Wang L, Schloss J A, Bonazzi V, McEwen J E, Wetterstrand K A, Deal C, Baker C C, Di Francesco V, Howcroft T K, Karp R W, Lunsford R D, Wellington C R, Belachew T, Wright M, Giblin C, David H, Mills M, Salomon R, Mullins C, Akolkar B, Begg L, Davis C, Grandison L, Humble M, Khalsa J, Little A R, Peavy H, Pontzer C, Portnoy M, Sayre M H, Starke-Reed P, Zakhari S, Read J, Watson B, Guyer M. The NIH Human Microbiome Project. Genome Res 2009; 19:2317-23.
8. Turnbaugh P J, Ley R E, Hamady M, Fraser-Liggett C M, Knight R, Gordon J I. The human microbiome project. Nature 2007; 449:804-10.
9. Round J L, Mazmanian S K. The gut microbiota shapes intestinal immune responses during health and disease. Nat Rev Immunol 2009; 9:313-23.
10. Ley R E, Peterson D A, Gordon J I. Ecological and evolutionary forces shaping microbial diversity in the human intestine. Cell 2006; 124:837-48.
11. Smith K, McCoy K D, Macpherson A J. Use of axenic animals in studying the adaptation of mammals to their commensal intestinal microbiota. Semin Immunol 2007; 19:59-69.
12. Ichinohe T, Pang I K, Kumamoto Y, Peaper D R, Ho J H, Murray T S, Iwasaki A. Microbiota regulates immune defense against respiratory tract influenza A virus infection. Proc Natl Acad Sci USA 2011; 108:5354-9.
13. Corthesy B, Gaskins H R, Mercenier A. Cross-talk between probiotic bacteria and the host immune system. J Nutr 2007; 137:781S-90S.
14. Akira S, Uematsu S, Takeuchi O. Pathogen recognition and innate immunity. Cell 2006; 124:783-801.
15. Harris G, KuoLee R, Chen W. Role of Toll-like receptors in health and diseases of gastrointestinal tract. World J Gastroenterol 2006; 12:2149-60.
16. Cambi A, Figdor C G. Levels of complexity in pathogen recognition by C-type lectins. Curr Opin Immunol 2005; 17:345-51.
17. Cambi A, Koopman M, Figdor C G. How C-type lectins detect pathogens. Cell Microbiol 2005; 7:481-8.
18. Dahl M R, Thiel S, Matsushita M, Fujita T, Willis A C, Christensen T, Vorup-Jensen T, Jensenius J C. MASP-3 and its association with distinct complexes of the mannan-binding lectin complement activation pathway. Immunity 2001; 15:127-35.
19. Ip W K, Takahashi K, Moore K J, Stuart L M, Ezekowitz R A. Mannose-binding lectin enhances Toll-like receptors 2 and 6 signaling from the phagosome. J Exp Med 2008; 205:169-81.
20. Kuronuma K, Sano H, Kato K, Kudo K, Hyakushima N, Yokota S, Takahashi H, Fujii N, Suzuki H, Kodama T, Abe S, Kuroki Y. Pulmonary surfactant protein A augments the phagocytosis of Streptococcus pneumoniae by alveolar macrophages through a casein kinase 2-dependent increase of cell surface localization of scavenger receptor A. J Biol Chem 2004; 279:21421-30.
21. Murakami S, Iwaki D, Mitsuzawa H, Sano H, Takahashi H, Voelker DR, Akino T, Kuroki Y. Surfactant protein A inhibits peptidoglycan-induced tumor necrosis factor-alpha secretion in U937 cells and alveolar macrophages by direct interaction with toll-like receptor 2. J Biol Chem 2002; 277:6830-7.
22. Takahashi H, Sano H, Chiba H, Kuroki Y. Pulmonary surfactant proteins A and D: innate immune functions and biomarkers for lung diseases. Curr Pharm Des 2006; 12:589-598.
23. Hontelez S, Sanecka A, Netea M G, van Spriel A B, Adema G J. Molecular view on PRR cross-talk in antifungal immunity. Cell Microbiol 2012; 14:467-74.
24. Ehrchen J M, Sunderkotter C, Foell D, Vogl T, Roth J. The endogenous Toll-like receptor 4 agonist S100A8/S100A9 (calprotectin) as innate amplifier of infection, autoimmunity, and cancer. J Leukoc Biol 2009; 86:557-66.
25. Wallin R P, Lundqvist A, More S H, von Bonin A, Kiessling R, Ljunggren H G. Heat-shock proteins as activators of the innate immune system. Trends Immunol 2002; 23:130-5.
26. Schaeffer L M, McCormack F X, Wu H, Weiss A A. Bordetella pertussis lipopolysaccharide resists the bactericidal effects of pulmonary surfactant protein A. J Immunol 2004; 173:1959-65.
27. Ragas A, Roussel L, Puzo G, Riviere M. The Mycobacterium tuberculosis cell-surface glycoprotein apa as a potential adhesin to colonize target cells via the innate immune system pulmonary C-type lectin surfactant protein A. J Biol Chem 2007; 282:5133-42.
28. Shi L, Takahashi K, Dundee J, Shahroor-Karni S, Thiel S, Jensenius J C, Gad F, Hamblin M R, Sastry K N, Ezekowitz R A. Mannose-binding lectin-deficient mice are susceptible to infection with Staphylococcus aureus. J Exp Med 2004; 199:1379-90.
29. Sato S, St-Pierre C, Bhaumik P, Nieminen J. Galectins in innate immunity: dual functions of host soluble beta-galactoside-binding lectins as damage-associated molecular patterns (DAMPs) and as receptors for pathogen-associated molecular patterns (PAMPs). Immunol Rev 2009; 230:172-87.
30. Isaksen B, Fagerhol M K. Calprotectin inhibits matrix metalloproteinases by sequestration of zinc. Mol Pathol 2001; 54:289-92.
31. Gout E, Moriscot C, Doni A, Dumestre-Perard C, Lacroix M, Perard J, Schoehn G, Mantovani A, Arlaud G J, Thielens N M. M-ficolin interacts with the long pentraxin PTX3: a novel case of cross-talk between soluble pattern-recognition molecules. J Immunol 2011; 186:5815-22.
32. Cash H L, Whitham C V, Behrendt C L, Hooper L V. Symbiotic bacteria direct expression of an intestinal bactericidal lectin. Science 2006; 313:1126-30.
33. van Ampting M T, Loonen L M, Schonewille A J, Konings I, Vink C, Iovanna J, Chamaillard M, Dekker J, van der Meer R, Wells J M, Bovee-Oudenhoven I M. Intestinally secreted C-type lectin Reg3b attenuates salmonellosis but not listeriosis in mice. Infect Immun 2012; 80:1115-20.
34. Kashyap D R, Wang M, Liu L H, Boons G J, Gupta D, Dziarski R. Peptidoglycan recognition proteins kill bacteria by activating protein-sensing two-component systems. Nat Med 2011; 17:676-83.
35. Tarkowski A, Bjersing J, Shestakov A, Bokarewa M I. Resistin competes with lipopolysaccharide for binding to toll-like receptor 4. J Cell Mol Med 2010; 14:1419-31.
36. Sands B E, Podolsky D K. The trefoil peptide family. Annu Rev Physiol 1996; 58:253-73.
37. Podolsky D K, Lynch-Devaney K, Stow J L, Oates P, Murgue B, DeBeaumont M, Sands B E, Mahida Y R. Identification of human intestinal trefoil factor. Goblet cell-specific expression of a peptide targeted for apical secretion. J Biol Chem 1993; 268:6694-702.
38. Yu A C, Worrall L J, Strynadka N C. Structural insight into the bacterial mucinase

StcE essential to adhesion and immune evasion during enterohemorrhagic E. coli infection. Structure 2012; 20:707-17.
39. Madsen J, Tornoe I, Nielsen O, Lausen M, Krebs I, Mollenhauer J, Kollender G, Poustka A, Skjodt K, Holmskov U. CRP-ductin, the mouse homologue of gp-340/deleted in malignant brain tumors 1 (DMBT1), binds gram-positive and gram-negative bacteria and interacts with lung surfactant protein D. Eur J Immunol 2003; 33:2327-36.
40. Pertoft H, Laurent T C, Laas T, Kagedal L. Density gradients prepared from colloidal silica particles coated by polyvinylpyrrolidone (Percoll). Anal Biochem 1978; 88:271-82.
41. Perkins D N, Pappin D J, Creasy D M, Cottrell J S. Probability-based protein identification by searching sequence databases using mass spectrometry data. Electrophoresis 1999; 20:3551-67.
42. Wisniewski J R, Zougman A, Nagaraj N, Mann M. Universal sample preparation method for proteome analysis. Nat Methods 2009; 6:359-62.
43. Pieper R, Zhang Q, Clark D J, Huang S T, Suh M J, Braisted J C, Payne S H, Fleischmann R D, Peterson S N, Tzipori S. Characterizing the Escherichia coli O157:H7 Proteome Including Protein Associations with Higher Order Assemblies. PLoS One 2011; 6:e26554.
44. Kuntumalla S, Zhang Q, Braisted J C, Fleischmann R D, Peterson S N, Donohue-Rolfe A, Tzipori S, Pieper R. In vivo versus in vitro protein abundance analysis of Shigella dysenteriae type 1 reveals changes in the expression of proteins involved in virulence, stress and energy metabolism. BMC Microbiol 2011; 11:147.
45. Pieper R, Zhang Q, Parmar P P, Huang S T, Clark D J, Alami H, Donohue-Rolfe A, Fleischmann R D, Peterson S N, Tzipori S. The Shigella dysenteriae serotype 1 proteome, profiled in the host intestinal environment, reveals major metabolic modifications and increased expression of invasive proteins. Proteomics 2009; 9:5029-45.
46. Apajalahti J H, Sarkilahti L K, Maki B R, Heikkinen J P, Nurminen P H, Holben W E. Effective recovery of bacterial DNA and percent-guanine-plus-cytosine-based analysis of community structure in the gastrointestinal tract of broiler chickens. Appl Environ Microbiol 1998; 64:4084-8.
47. van der Burg M P, Graham J M. Iodixanol density gradient preparation in university of wisconsin solution for porcine islet purification. ScientificWorldJournal 2003; 3:1154-9.

1. A method for analyzing a complex host-microbial mixture, comprising the steps of:
(a) fractionating the complex host-microbial mixture to obtain insoluble cellular and sub-cellular aggregates;
(b) preparing a protein mixture from the aggregates; and
(c) performing a metaproteomic analysis of the mixture.
